(19) United States
(12) Patent Application Publication
(10) Pub. No.: US 2014/0155777 A1
(43) Pub. Date: Jun. 5, 2014

(54) MOUTHPIECE FOR BREATHALYZER

(71) Applicant: Cheol-Gyu Kong, Gwangju (KR)

(72) Inventor: Cheol-Gyu Kong, Gwangju (KR)

(73) Assignee: YOUN-JEONG JANG, Gwangju (KR)

(21) Appl. No.: 14/232,835

(22) PCT Filed: Jul. 15, 2012
(86) PCT No.: PCT/KR2012/005639

§ 371 Date: Jan. 14, 2014

(30) Foreign Application Priority Data
Jul. 16, 2011 (KR) 20-2011-0006474

(51) Int. Cl. A61B 5/097; G01N 33/497; A61B 5/08

(52) U.S. Cl. CPC A61B 5/097; A61B 5/082; G01N 33/4972
USPC 600/532

(57) ABSTRACT

A mouthpiece for a breathalyzer. A body (20) includes a breath inlet (22) and a breath outlet (24) through which breath enters and exits. A breathalyzer fitting part (30) protrudes from the outer circumference of the body (20), and is fitted into a breathalyzer (10). A transparent hollow breath indicator (40) is disposed on the body (20) so as to be perpendicular to the lengthwise direction of the body (20). A breath flow hole (50) provided on the body (20) allows the breath indicator (40) and the body (20) to communicate with each other such that the breath that has entered through the breath inlet (22) moves into the breath indicator (40). A breath indicator ball (60) is movable upward and downward inside the breath indicator (40) so as to be lifted up by the breath that has entered the breath indicator (40) through the breath flow hole (50).

### CROSS-REFERENCE TO RELATED APPLICATION

This application claims the benefit of Korean Patent Application No. 20-2011-0006474, filed on Jul. 16, 2011 in the Korean Intellectual Property Office, the disclosure of which is incorporated herein by reference.

### TECHNICAL FIELD

The present invention relates to a mouthpiece for a breathalyzer, and more particularly, to a breathalyzer mouthpiece which is designed to measure the amount of breath when a driver blows the breath into the breathalyzer.

### BACKGROUND ART

In general, a variety of car accidents occur due to rapid distribution of cars. Among these accidents, the number of drunk driving accidents is increasing day by day. Therefore, governments are enforcing a variety of prevention programs in order to prevent drunk driving.

As a measure of such drunk driving prevention programs, breathalyzers are used in order to measure the blood alcohol content of drivers at sites. Such a breathalyzer is a device that indicates the blood alcohol content of a driver as a data when the driver puts his/her mouth to a mouthpiece of the breathalyzer and blows air into the breathalyzer through the mouthpiece. This device determines whether or not the driver is drunk and how much the driver drunk based on the blood alcohol content in the air blown out while he/she is breathing.

However, drivers are mentally reluctant to blood alcohol content measurement, and in most cases, act non-cooperatively. For example, during the blood alcohol content measurement, drivers blow a small amount of air or pretend to blow air into the breathalyzer through the mouthpiece, or momentarily breathe in during the exhalation of air.

Therefore, there is frequent friction between drivers and measuring personnel. Since there is frequent friction between drivers who are reluctant to the blood alcohol content measurement and the measuring personnel (traffic police), there is required a better blood alcohol content measurement method that can cope with the non-cooperative behavior of drivers.

A related-art mouthpiece used in such a breathalyzer is shown in FIG. 1. The mouthpiece shown in FIG. 1 is disclosed in Korean Registered Utility Model No. 0126889 (titled “MOUTHPIECE FOR BREATHALYZER”). However, since the mouthpiece is configured as a simple passage through which air passes, when a driver acts non-cooperatively as described above, it was impossible to visually recognize whether or not the driver has blown air through the mouthpiece. In some cases, accurate measurement was difficult since air that has entered the breathalyzer flowed back or the amount of the air was insufficient for measurement.

In addition, even if the driver has blown out air, the state in which the air is entering the breathalyzer is not indicated. Thus, measurement depended on the sense of the measuring personnel, which is problematic.

### DISCLOSURE

### Technical Problem

The present invention has been made to solve the foregoing problems with the related art, and therefore an object of the present invention is to provide a breathalyzer mouthpiece which allows the amount of breath blown out by a driver to be visually recognized when the driver acts non-cooperatively in measurement.

### Technical Solution

In order to realize the foregoing object of the present invention, provided is a breathalyzer mouthpiece that includes: a body including a breath inlet through which breath enters and a breath outlet through which the breath exits; a breathalyzer fitting part protruding from an outer circumferential portion of the body, the breathalyzer fitting part being fitted into a breathalyzer; a transparent hollow breath indicator disposed on an outer circumference of the body so as to be perpendicular to a lengthwise direction of the body; and a breath flow hole provided on the body. The breath flow hole allows the breath indicator and the body to communicate with each other such that the breath that has entered through the breath inlet moves into the breath indicator. The breathalyzer mouthpiece further includes a breath indicator ball. The breath indicator ball is movable in an upward-downward direction inside the breath indicator so as to be lifted up by the breath that has entered the breath indicator through the breath flow hole.

Here, the breath inlet may be provided in one end of the body, and the breath outlet may be provided in the other end or an outer circumferential portion of the body.

The breath indicator may include numerical value indicator lines. The numerical value indicator lines are arranged at predetermined intervals in order to indicate lifted positions of the breath indicator ball. The breath indicator may also include a numerical value reference line which indicates whether or not an amount of breath required for an operation of the breathalyzer has entered.

The breath indicator ball may be painted with or made of a fluorescent material so as to be easily recognizable at night.

The inner diameter of the breath outlet may be smaller than an inner diameter of the breath inlet. The body may have a fitting hole (26) through which the breath enters the breathalyzer fitting part (30). The inner diameter of each of the fitting hole (26) and the breath flow hole (50) may be smaller than an inner diameter of the breath outlet

### Advantageous Effects

The breathalyzer mouthpiece according to the invention as set forth above can visually indicate whether or not a driver has blown breath, thereby fundamentally eliminating friction between the driver and the measuring personnel.

Furthermore, since the breath indicator ball is painted with or made of a fluorescent material so as to be recognized at night, the breath indicator ball can be easily recognized during blood alcohol content measurement.

### DESCRIPTION OF DRAWINGS

FIG. 1 is a configuration view showing a breathalyzer mouthpiece of the related art;

FIG. 2 is a perspective view showing a breathalyzer mouthpiece according to an embodiment of the invention;

FIG. 3 is a cross-sectional view of FIG. 2;

FIG. 4 is a cross-sectional view showing the state in which a breath indicator ball is lifted up by breath that is blown into the breathalyzer mouthpiece according to an embodiment of the invention;

FIG. 5 is a view showing the state in which the breathalyzer mouthpiece according to an embodiment of the invention is mounted on a breathalyzer;

FIG. 6 is a perspective view showing a breathalyzer mouthpiece according to another embodiment of the invention;

FIG. 7 is a cross-sectional view of FIGS. 6; and

FIG. 8 is a cross-sectional view showing another form of a breath outlet in the breathalyzer mouthpiece according to another embodiment of the invention.

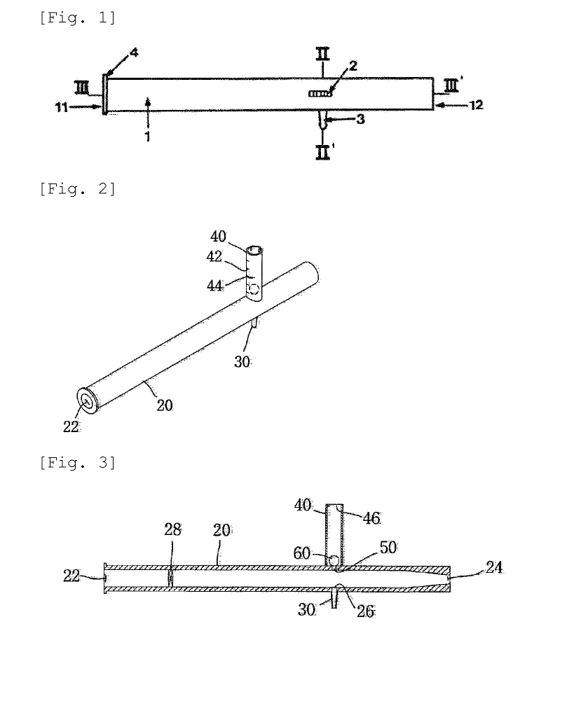

### BEST MODE

Reference will now be made in detail to exemplary embodiments of a breathalyzer mouthpiece according to the invention, examples of which are illustrated in the accompanying drawings.

FIG. 2 is a perspective view showing a breathalyzer mouthpiece according to an embodiment of the invention, and FIG. 3 is a cross-sectional view of FIG. 2. The breathalyzer mouthpiece according to an embodiment of the invention includes a body 20 and a breathalyzer fitting part 30. The body 20 has a breath inlet 22 on one end through which breath enters and a breath outlet 24 on the other end through which the breath exits, the breath outlet 24 communicating with the breath inlet 22. The breathalyzer fitting part 30 protrudes from an outer circumferential portion of the body 20 such that it can be fitted into a breathalyzer 10. The breathalyzer mouthpiece further includes a breath indicator 40, a breath flow hole 50 and a breath indicator ball 60 in order to indicate whether or not a driver has blown breath into the body 20.

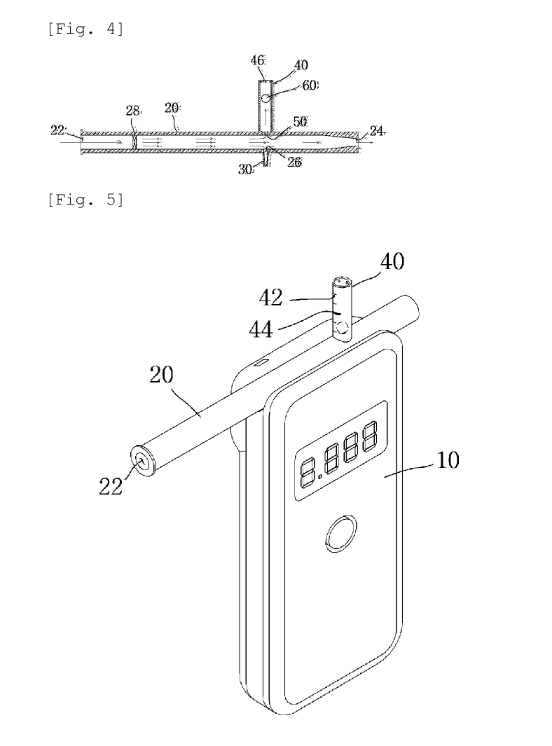

The body 20 has the shape of a hollow pipe having a preset length. According to an embodiment, as shown in FIG. 3 and FIG. 4, the breath inlet 22 and the breath outlet 24 are respectively provided on one end and the other end of the body 20. When breath enters through the breath inlet, a portion of the breath is introduced through a fitting hole 26 which communicates with the breath flow hole 50 and the breathalyzer fitting part 30, and the remaining portion of the breath is discharged to the outside through the breath outlet 24.

In addition, according to another embodiment, as shown in FIG. 6 and FIG. 7, the breath inlet 22 is provided on one end, and the breath outlet 24 is provided on an outer circumferential portion of the body 20. In the accompanying figures, the breath outlet 24 is formed between the breath inlet and the breathalyzer fitting part 30 so as to extend upward.

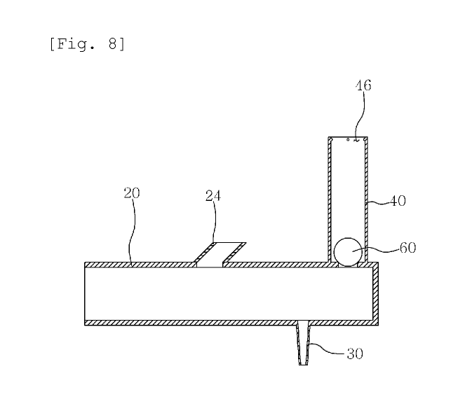

When the breath outlet is formed to extend upward through the outer circumference of the body, it is preferred that the end portion thereof be bent, as shown in FIG. 7, or inclined, as shown in FIG. 8, so as to face away from the breath inlet so that breath discharged therefrom does not reach the driver.

Here, the breath outlet 24 is configured such that the inner diameter thereof is smaller than that of the breath inlet 22 so that a portion of the breath that has entered through the breath inlet flows into the hole of the fitting part.

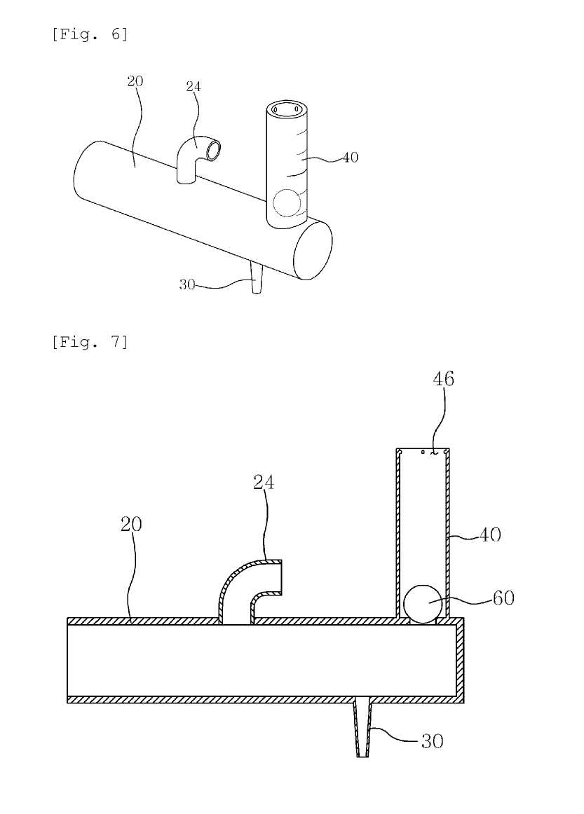

The breath indicator 40 is implemented as a transparent hollow body in order to provide visual indication to the driver or measuring personnel, and is provided at the opposite side from the breathalyzer fitting part 30. As shown in FIG. 5, when the breathalyzer mouthpiece according to the invention is installed in a breathalyzer, the breath indicator 40 is positioned opposite the breathalyzer.

The breath indicator 40 is disposed at the other side of the outer circumference of the body 20 that is opposite the breathalyzer fitting part 30, and is oriented perpendicular to the lengthwise direction of the body 20. Although the breath indicator can be disposed at an incline, it is preferred that the breath indicator be oriented perpendicularly so that the breath indicator ball can move and be recognized properly.

Numerical value indicator lines 42 are formed on the breath indicator 40 at preset intervals in order to indicate the lifted position of the breath indicator ball 60 when the breath indicator ball 60 is lifted up by the breath that has entered the breath indicator 40. Among the numerical value indicator lines 42, a numerical value reference line 44 indicates whether or not an amount of breath required for the operation of the breathalyzer has entered.

Therefore, when the breath indicator ball 60 is lifted up to or above the numerical value reference line 44 by an amount of breath that has been blown out by the driver, it can be appreciated that the breath has entered as much as required for the operation of the breathalyzer.

Of course, the height of the numerical value reference line 44 is determined in consideration of the size of the breath flow hole 50, the weight of the breath indicator ball, or the like at the time of fabrication.

The breath flow hole 50 is formed in the body 20 such that breath can flow into the breath indicator 40. The breath flow hole 50 allows the breath indicator 40 and the inside of the body 20 to communicate with each other such that the breath that has entered through the breath inlet 22 can flow into the breath indicator.

The breath flow hole 50 is configured such that an excessive amount of breath does not pass through the breath flow hole. The breath flow hole 50 can be configured such that the inner diameter thereof can be adjusted. Here, it is preferred that the diameter of each of the breath flow hole 50 and the fitting hole 26 formed in the body 20 be smaller than that of the breath outlet 24.

Accordingly, a majority of the breath that has entered through the breath inlet 22 is discharged through the breath outlet 24, and only a portion of the breath can flow into the breath flow hole and the fitting hole.

The breath indicator ball 60 is provided such that it can move freely inside the breath indicator 40. The breath indicator ball 60 is lifted up by breath that has flown into the breath indicator 40 through the breath flow hole 50 or moves downward as the breath has leaked.

Here, when the breath indicator ball 60 is lifted up, air inside the breath indicator 40 above the breath indicator ball 60 is discharged to the outside through a top hole 46 in the upper portion. When the breath indicator ball 60 has moved down, air inside the breath indicator below the breath indicator ball 60 leaks through a fine gap between the breath indicator ball and the inner circumference of the breath indicator and the breath flow hole.

In addition, it is preferred that the breath indicator ball 60 be painted with a noticeable color, such as yellow or red, instead of being transparent or being painted with white such that the breath indicator ball 60 can be easily recognized not only in the daytime but also at night. In particular, the breath indicator ball can be painted with or made of a fluorescent material such that the breath indicator ball can be easily recognized at night.

In the meantime, a mesh 28 is provided inside the body 20 to be adjacent to the breath inlet 22. The mesh 28 causes the speed of the breath that flows into the body 20 through the breath inlet 22 to be uniform.

In the case of measuring blood alcohol content by the breathalyzer mouthpiece according to the invention as configured above to a breathalyzer, breath from the lungs of a driver is blown out through the breath inlet. Then, as shown in FIG. 4, one portion of the breath enters through the fitting hole, another portion of the breath enters through the breath flow hole, and the remaining portion of the breath exits through the breath outlet.

At this time, the breath indicator ball 60 moves upward inside the breath indicator by being lifted up by the air that has flown into the breath indicator through the breath flow hole.

Accordingly, the breath indicator ball allows the driver to visually recognize the amount of the breath that he/she has blown out. When the breath indicator ball has not moved above the reference line, the driver must blow breath again. This can consequently prevent the driver from evading alcohol measurement. Otherwise, the driver would have not cooperated in a variety of methods.

Accordingly, when the driver blows breath through the mouthpiece, the amount of breath that the driver has blown out can be recognized. This can consequently minimize friction between the driver and measuring personnel which would otherwise occur frequently, while helping to eliminate distrust in breathalyzers.

1. A breathalyzer mouthpiece comprising:
a body (20) comprising a breath inlet (22) through which breath enters and a breath outlet (24) through which the breath exits;
a breathalyzer fitting part (30) protruding from an outer circumferential portion of the body (20), the breathalyzer fitting part (30) being fitted into a breathalyzer (10);
a transparent hollow breath indicator (40) disposed on an outer circumference of the body (20) so as to be perpendicular to a lengthwise direction of the body (20);
a breath flow hole (50) provided on the body (20), wherein the breath flow hole (50) allows the breath indicator (40) and the body (20) to communicate with each other such that the breath that has entered through the breath inlet (22) moves into the breath indicator (40); and
a breath indicator ball (60), wherein the breath indicator ball (60) is movable in an upward-downward direction inside the breath indicator (40) so as to be lifted up by the breath that has entered the breath indicator (40) through the breath flow hole (50).

2. The breathalyzer mouthpiece according to claim 1, wherein the breath inlet (22) is provided in one end of the body (20), and the breath outlet (24) is provided in the other end or an outer circumferential portion of the body (20).

3. The breathalyzer mouthpiece according to claim 1, wherein the breath indicator (40) comprises numerical value indicator lines (42), wherein the numerical value indicator lines (42) are arranged at predetermined intervals in order to indicate lifted positions of the breath indicator ball (60), and a numerical value reference line (44) which indicates whether or not an amount of breath required for an operation of the breathalyzer (10) has entered.

4. The breathalyzer mouthpiece according to claim 1, wherein the breath indicator ball (60) is painted with or made of a fluorescent material so as to be easily recognizable at night.

5. The breathalyzer mouthpiece according to claim 1, wherein an inner diameter of the breath outlet (24) is smaller than an inner diameter of the breath inlet (22), and wherein the body (20) has a fitting hole (26) through which the breath enters the breathalyzer fitting part (30), an inner diameter of each of the fitting hole (26) and the breath flow hole (50) being smaller than an inner diameter of the breath outlet (24).

6. The breathalyzer mouthpiece according to claim 2, wherein the breath indicator (40) comprises numerical value indicator lines (42), wherein the numerical value indicator lines (42) are arranged at predetermined intervals in order to indicate lifted positions of the breath indicator ball (60), and a numerical value reference line (44) which indicates whether or not an amount of breath required for an operation of the breathalyzer (10) has entered.
